(19) United States
(12) Patent Application Publication
(10) Pub. No.: US 2026/0367077 A1
(43) Pub. Date: Nov. 19, 2026

(54) ELECTROCHEMICAL DETECTION OF AEROSOLIZED PATHOGENS USING AN IMMUNE-BASED BIOSENSOR

(71) Applicant: Washington University, St. Louis, MO (US)

(72) Inventors: John CIRRITO, St. Louis, MO (US); Carla YUEDE, St. Louis, MO (US); Rajan CHAKRABARTY, St. Louis, MO (US)

(21) Appl. No.: 19/158,743

(22) PCT Filed: Feb. 19, 2024
(86) PCT No.: PCT/US24/16359

§ 371 Date: Aug. 21, 2025

Related U.S. Application Data
(60) Provisional application No. 63/486,282, filed on Feb. 22, 2023.

(51) Int. Cl. G01N 33/543; A61B 5/08; A61B 5/097; G01N 27/327; G01N 27/48; G01N 33/569

(52) U.S. Cl. CPC G01N 33/5438; A61B 5/082; A61B 5/097; G01N 27/3278; G01N 27/48; G01N 33/56983; G01N 2333/165; G01N 2469/10

(57) ABSTRACT

The present disclosure is directed to a biosensor, a method, and an airborne detection device for analyzing aerosolized samples from breath or an environmental air sample to detect aerosolized pathogens. The biosensor includes an activated electrode including a nanobody deposited thereon. The airborne detection device further includes a sample collection device.

### RELATED APPLICATIONS

This application claims priority to U.S. Provisional Application Ser. No. 63/486,282, filed on Feb. 22, 2023, the contents of which are hereby incorporated by reference in their entirety.

### STATEMENT REGARDING FEDERALLY SPONSORED RESEARCH & DEVELOPMENT

This invention was made with government support under grant number U01AA029331 awarded by the National Institutes of Health. The government has certain rights in the invention.

### FIELD OF THE DISCLOSURE

The field of the disclosure relates generally to biosensors, methods, and devices for analyzing aerosolized samples from breath or an environmental air sample to detect aerosolized pathogens. More specifically, the biosensor includes an activated electrode including a nanobody deposited thereon. The airborne detection device further includes a sample collection device.

### BACKGROUND OF THE DISCLOSURE

Inhalation of virus-laden aerosols exhaled by infected individuals is deemed as a primary transmission mode of respiratory viruses such as SARS-CoV-2, influenza virus, rhinovirus, and respiratory syncytial virus (RSV). Respiratory emissions during infection show the presence of viral RNA in a variety of aerosol sizes, with higher viral loads detected in aerosols <1 μm compared to larger size aerosols. Sub-micrometer size virus aerosols are predominantly produced during breathing. The production of these aerosols involve bursting of the fluid film in respiratory bronchioles in the lower airways of a human lung. Despite the demonstrated significance of disease transmission via aerosols, techniques for direct, real-time detection of respiratory virus aerosols have remained elusive.

SARS-CoV-2 viral genomes have been reported to be highest in the sub-micrometer size range of aerosols exhaled by infected individuals. Screening non-invasively for SARS-CoV-2 viral RNA in breath aerosols remains a technical challenge. Current state of research involves exhaled breath condensate (EBC) collection followed by reverse transcription-polymerase chain reaction (RT-PCR) to detect the prevalence of SARS-CoV-2 in various communities. This methodology has limitations for mass testing applications due to long turnaround times (ranging from few hours to days) and the need for sophisticated equipment and trained personnel. Indirect methods of detection using electrochemical sensors have also been introduced to quantify the volatile organic compounds (VOCs) in exhaled breath that are associated with COVID-19. These techniques discern a distinct pattern or signature of VOC emissions corresponding to a particular variant. Thus, their feasibility might be hindered with the emergence of new SARS-CoV-2 variants. The situation underscores the need for variant-sensitive and rapid testing solutions at the point-of-need for early intervention and prevention of the community spread of the disease.

Accordingly, there is a need for direct, rapid disposable, single-use, low-cost, detection techniques and tests that are envisioned for scalable production and use in hospitals, airports, schools, and anywhere where a large number of people are expected to gather. In particular, there is a need for electrochemical methods that offer improved limits of detection and higher fidelity than currently available “rapid” antigen tests. The embodiments described herein resolve at least these known deficiencies.

### BRIEF DESCRIPTION OF THE DISCLOSURE

In one aspect, the present disclosure is directed to an airborne detection device for analyzing aerosolized samples from breath or an environmental air sample to detect aerosolized pathogens, the device comprising: an analysis vial; and a biosensor electrode.

In another aspect, the present disclosure is directed to a method for detecting aerosolized pathogens, the method comprising: transporting a liquid sample from an external sampling device to an analysis vial of an aerosol collection device; adding working fluid to the analysis vial of the aerosol collection device; detecting at least one pathogen; replenishing sample fluid to the external sampling device; and evacuating an analyzed sample from the analysis vial to a waste reservoir of the aerosol collection device.

In yet another aspect, the present disclosure is directed to a system for detecting airborne pathogens, the system comprising: an airborne detection device; and an external sampling device.

In some embodiments, multiple pathogens are detectable simultaneously in a single test, wherein the multiple pathogens are selected from combinations of viruses, bacteria, parasites, fungi, and mold, or multiple viruses, multiple bacteria, or multiple species or strains of viruses, bacteria, parasites, fungi, or mold. In some embodiments, multiple variants of a pathogen are detectable from a single test, and wherein the multiple variants include delta and omicron variants of SARS-CoV-2. In some embodiments, multiple pathogens are detected simultaneously in a multiplex test.

### BRIEF DESCRIPTION OF THE DRAWINGS

The embodiments described herein may be better understood by referring to the following description in conjunction with the accompanying drawings.

FIG. 1 is an exemplary embodiment of a breath aerosol analyzer system in accordance with the present disclosure. Depictions include sampling of breath aerosols generated from the lower respiratory tract during normal breathing, a schematic of the breath aerosol analyzer system including an aerosol collector, a MIE biosensor and a potentiostat module, illustration of the mechanism of virus detection using the MIE biosensor, and a picture of the 3D-printed breath aerosol collection box and the cap with an inlet straw.

FIG. 2A is an exemplary embodiment of the specificity of the MIE biosensor tested with SARS-CoV-1 and SARS-CoV-2 spike protein in accordance with the present disclosure.

FIG. 2B is an exemplary embodiment of the biosensor sensitivity (or LoD), which was evaluated by serial dilution of different SARS-CoV-2 variants in accordance with the present disclosure.

FIG. 2C is an exemplary embodiment in accordance with the present disclosure depicting left) Normalized oxidation current (Iₒₓ) measured by the MIE biosensor in laboratory experiments, where the horizontal dashed line denotes the limit of detection (LoD) of the system, and right) Viral RNA copies/mL determined using RT-qPCR for different aerosolized SARS-CoV-2 variants. The differences between viral RNA copies obtained for the four SARS-CoV-2 variants were statistically insignificant (t-test, p=0.17) indicating that the strain of virus did not impact the virus collection efficiency of the breath aerosol collection device. Whiskers denote the range of data, and box represents the inter-quartile range.

FIG. 3A is an exemplary embodiment of an estimate of the minimum number of exhaled breaths for detection by the MIE biosensor, with the number of exhaled breaths predicted by evaluating the viral copies per breath for assumed range of viral load for COVID-19 patients in accordance with the present disclosure.

FIG. 3B is an exemplary embodiment of an estimate of the minimum number of exhaled breaths for detection by the MIE biosensor, with clinical study results demonstrating that SARS-CoV-2 viral particles are detected in as low as two exhaled breaths of patients.

FIG. 4 is an exemplary embodiment of an experimental setup for aerosolization of SARS-CoV-2 variants in accordance with the present disclosure. Compressed air at a pressure of 20 psi from the CHεST® and inactivated SARS-CoV-2 virus solution are sent to the BLAM atomizer. The atomizer generates stream of aerosolized particles which is constricted using a conical attachment for enhanced impaction on the chilled breathalyzer surface. Virus-laden aerosols condense and trickle down, and EBC sample is collected from the bottom of the breath aerosol collector.

FIG. 5A is an exemplary embodiment of a probability density function (PDF) of virus aerosols in accordance with the present disclosure. Using different configurations of the CHεST® setup, virus-laden aerosols of different sizes under the sub-micron range (<1 μm) were produced to mimic 10-15 “deep” exhaled breaths by a person.

FIG. 5B is an exemplary embodiment of modeling of the EBC breathalyzer collection efficiency at different temperatures of the impact surface in accordance with the present disclosure. The collection efficiency of EBC by the breathalyzer is highly dependent on the temperature differential between the warm exhaled breath and the temperature of the surface blown on. The breath collection was modeled at different temperatures of the hydrophobic impact surface. At 4 °C efficiency should be ~18.6% whereas at −20 °C there is a 43.6% recovery of aerosol particles by the EBC.

FIG. 6 is an exemplary embodiment of virus aerosol recovery (%) for different variants after aerosolization and condensation using a collection device in accordance with the present disclosure. The range in virus aerosol recovery (%) values for the different variants is dependent on factors like the volume of EBC sample collected and viral RNA degradation due to delay in sample processing from the time of collection. A similar spread in data is also observed in the recovery of test fluids using other commercial EBC collection devices.

FIG. 7 is an exemplary embodiment of normalized Ix values for the clinical data of 8 patients in accordance with the present disclosure. Columns with thick borders indicate false negative results.

FIG. 8 is an exemplary embodiment of SARS-CoV-2 biosensor design in accordance with the present disclosure. The CoV-2 biosensor uses square wave voltammetry to measure oxidation of tyrosine amino acids within the viral particle. Oxidation releases electrons that the sensor detects as current. The biosensor uses a nanobody attached to the surface to provide specificity and concentrate the viral particle at the electrode for measurement. The electrode is blocked with albumin to limit non-specific signal.

FIG. 9 is an exemplary embodiment of Aβ MIE biosensors used in the brains of living mice in accordance with the present disclosure. ISF Aβ₄₀ concentrations over 180 minutes in APP/PS1 (n=6) and WT (n=3) mice.

### DETAILED DESCRIPTION OF THE DISCLOSURE

The present disclosure describes a biosensor that is deployed in a breathalyzer or an environmental sensor device that detects aerosolized virus within a breath or given space (such as any indoor or enclosed space, including large spaces or other spaces having a potentially shared airspace) to determine if viral particles (such as aerosolized CoV-2) are present. Also described is an electrochemical, antibody-based biosensor to detect inactivated viral particles of at least one respiratory virus (or several respiratory viruses) alternatively or additional to SARS-CoV-2 virus particles. In some embodiments, the biosensor detects at least one bacterial genus or species. In other embodiments, the device detects at least one parasite, fungi, or mold genus or species.

According to the present disclosure, significant progress has been made in the development of a device for real-time detection of aerosolized pathogens (including SARS-CoV-2) using an immuno-based biosensor. The device is primarily for rapid testing of infected individuals (both symptomatic and asymptomatic) at a diagnostic level within 60 seconds of breathing into the device with the expectation that follow-up testing is not required. A single-use disposable exhaled breath condensate (EBC) collection device is used for capturing breath aerosols. The patient blows into the collection device which includes a cooled (~10 °C) hydrophobic film on the interior. The temperature difference between the exhaled breath and cool surface leads to condensation-based growth and collection of aerosol particles. Test buffer is added to collect the breath condensate which is applied to the biosensor for detection. In some embodiments, the breath condensate is applied to the biosensor and subsequently analyzed within several seconds or minutes of addition of the test buffer. In other embodiments, an exhaled breath sample may be collected (e.g., condensed and washed with test buffer) and suitably stored for a period of time (e.g., hours, days, weeks, etc.) prior to contact with the biosensor for pathogen detection. For instance, remote or at-home testing EBC collection devices may be provided such that collected samples require storage and/or transportation prior to contact with a biosensor for analysis. Depending on the embodiment, the breathalyzer is deployable to hospitals, schools, airports, and military facilities/vessels where a long queue of individuals needs to be rapidly tested. These devices will provide rapid readouts and act as a platform to detect other respiratory viruses and emerging pathogens.

Viral particles detectable by the disclosed biosensor include, but are not limited to, viruses associated with Chikungunya, Cholera, Crimean-Congo hemorrhagic fever, Ebola virus disease, Hendra virus infection, Influenza (pandemic, seasonal, zoonotic), Lassa fever, Marburg virus disease, Meningitis, MERS-CoV, Monkeypox, Nipah virus infection, Novel coronavirus (2019-nCoV), Plague, Rift Valley fever, SARS, Smallpox, Tularaemia, Yellow fever, Zika virus disease, Ebola and Marburg virus (Filoviridae); Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichinde virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); influenza A virus (Orthomyxoviridae); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Ebola and Marburg virus (Filoviridae); Semliki Forest virus, Ross River virus, chikungunya virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Junin virus, Machupo virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); human immunodeficiency virus type 1 (Retroviridae, Lentivirus); influenza A/B virus (Orthomyxoviridae, the common ‘flu’ virus); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); Variola major (smallpox) virus (Poxviridae, Chordopoxvirinae, Orthopoxvirus); human hepatitis B virus (Hepadnaviridae, Orthohepadnavirus); hepatitis delta virus (hepatitis D virus) (unassigned Family, Deltavirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus), Adeno-associated virus Dependovirus, Parvoviridae Aichi virus Kobuvirus, Picornaviridae Australian bat lyssavirus, Rhabdoviridae BK polyomavirus, Polyomaviridae Banna virus Seadornavirus, Reoviridae Barmah forest virus Alphavirus, Togaviridae Bunyamwera virus Orthobunyavirus, Bunyaviridae Bunyavirus La Crosse Orthobunyavirus, Bunyaviridae Bunyavirus snowshoe hare Orthobunyavirus, Bunyaviridae Cercopithecine herpesvirus Lymphocryptovirus, Herpesviridae Chandipura virus Vesiculovirus, Rhabdoviridae Chikungunya virus Alphavirus, Togaviridae Cosavirus A Cosavirus, Picornaviridae Cowpox virus Orthopoxvirus, Poxviridae Coxsackievirus Enterovirus, Picornaviridae Crimean-Congo hemorrhagic Nairovirus, Bunyaviridae fever virus Dengue virus Flavivirus, Flaviviridae Dhori virus Thogotovirus, Orthomyxoviridae Dugbe virus Nairovirus, Bunyaviridae Duvenhage virus Lyssavirus, Rhabdoviridae Eastern equine encephalitis virus Alphavirus, Togaviridae Ebolavirus, Filoviridae Echovirus Enterovirus, Picornaviridae Encephalomyocarditis virus Cardiovirus, Picornaviridae Epstein-Barr virus Lymphocryptovirus, Herpesviridae European bat lyssavirus, Rhabdovirus GB virus C/Hepatitis G virus Pegivirus, Flaviviridae Hantaan virus Hantavirus, Bunyaviridae Hendra virus Henipavirus, paramyxoviridae Hepatitis A virus Hepatovirus, picornaviridae Hepatitis B virus Orthohepadnavirus, Hepadnaviridae Hepatitis C virus Hepacivirus, Flaviviridae Hepatitis E virus Hepevirus, Unassigned Hepatitis delta virus Deltavirus, Unassigned Horsepox virus Orthopoxvirus, Poxviridae Human adenovirus Mastadenovirus, Adenoviridae Human astrovirus Mamastrovirus, Astroviridae Human coronavirus Alphacoronavirus, Coronaviridae Human cytomegalovirus, Herpesviridae Human enterovirus 68, 70 Enterovirus, Picornaviridae Human herpesvirus 1 Simplexvirus, Herpesviridae Human herpesvirus 2 Simplexvirus, Herpesviridae Human herpesvirus 6 Roseolovirus, Herpesviridae Human herpesvirus 7 Roseolovirus, Herpesviridae Human herpesvirus 8 Rhadinovirus, Herpesviridae Human immunodeficiency virus Lentivirus, Retroviridae Human papillomavirus 1 Mupapillomavirus, Papillomaviridae Human papillomavirus 2 Alphapapillomavirus, Papillomaviridae Human papillomavirus 16, 18 Alphapapillomavirus, Papillomaviridae Human parainfluenza Respirovirus, Paramyxoviridae Human parvovirus B19 Erythrovirus, Parvoviridae Human respiratory syncytial virus Orthopneumovirus, Pneumoviridae Human rhinovirus Enterovirus, Picornaviridae Human SARS coronavirus Betacoronavirus, Coronaviridae Human spumaretrovirus Spumavirus, Retroviridae Human T-lymphotropic virus Deltaretrovirus, Retroviridae Human torovirus, Coronaviridae Influenza A virus Influenzavirus A, Orthomyxoviridae Influenza B virus Influenzavirus B, Orthomyxoviridae Influenza C virus Influenzavirus C, Orthomyxoviridae Isfahan virus Vesiculovirus, Rhabdoviridae JC polyomavirus, Polyomaviridae Japanese encephalitis virus Flavivirus, Flaviviridae Junin arenavirus, Arenaviridae KI Polyomavirus, Polyomaviridae Kunjin virus Flavivirus, Flaviviridae Lagos bat virus Lyssavirus, Rhabdoviridae Lake Victoria marburgvirus Marburgvirus, Filoviridae Langat virus Flavivirus, Flaviviridae Lassa virus Arenavirus, Arenaviridae Lordsdale virus Norovirus, Caliciviridae Louping ill virus Flavivirus, Flaviviridae Lymphocytic choriomeningitis Arenavirus, Arenaviridae virus Machupo virus Arenavirus, Arenaviridae Mayaro virus Alphavirus, Togaviridae MERS coronavirus Betacoronavirus, Coronaviridae Measles virus Morbilivirus, Paramyxoviridae Mengo encephalomyocarditis virus Cardiovirus, Picornaviridae Merkel cell polyomavirus, Polyomaviridae Mokola virus Lyssavirus, Rhabdoviridae Molluscum contagiosum virus Molluscipoxvirus, Poxviridae Monkeypox virus Orthopoxvirus, Poxviridae Mumps virus Rubulavirus, Paramyxoviridae Murray valley encephalitis virus Flavivirus, Flaviviridae New York virus Hantavirus, Bunyavirus Nipah virus Henipavirus, Paramyxoviridae Norwalk virus Norovirus, Caliciviridae O'nyong-nyong virus Alphavirus, Togaviridae Orf virus Parapoxvirus, Poxviridae Oropouche virus Orthobunyavirus, Bunyaviridae Pichinde virus Arenavirus, Arenaviridae Poliovirus Enterovirus, Picornaviridae Punta toro phlebovirus, Bunyaviridae Puumala virus Hantavirus, Bunyavirus Rabies virus Lyssavirus, Rhabdoviridae Rift valley fever virus Phlebovirus, Bunyaviridae Rosavirus A Rosavirus, Picornaviridae Ross river virus Alphavirus, Togaviridae Rotavirus A Rotavirus, Reoviridae Rotavirus B Rotavirus, Reoviridae Rotavirus C Rotavirus, Reoviridae Rubella virus Rubivirus, Togaviridae Sagiyama virus Alphavirus, Togaviridae Salivirus A Salivirus, Picornaviridae Sandfly fever sicilian virus Phlebovirus, Bunyaviridae Sapporo virus Sapovirus, Caliciviridae Semliki forest virus Alphavirus, Togaviridae Seoul virus Hantavirus, Bunyavirus Simian foamy virus Spumavirus, Retroviridae Simian virus 5 Rubulavirus, Paramyxoviridae Sindbis virus Alphavirus, Togaviridae Southampton virus Norovirus, Caliciviridae St. louis encephalitis virus Flavivirus, Flaviviridae Tick-borne powassan virus Flavivirus, Flaviviridae Torque teno virus Alphatorquevirus, Anelloviridae Toscana virus Phlebovirus, Bunyaviridae Uukuniemi virus Phlebovirus, Bunyaviridae Vaccinia virus Orthopoxvirus, Poxviridae Varicella-zoster virus Varicellovirus, Herpesviridae Variola virus Orthopoxvirus, Poxviridae Venezuelan equine encephalitis Alphavirus, Togaviridae virus Vesicular stomatitis virus Vesiculovirus, Rhabdoviridae Western equine encephalitis virus Alphavirus, Togaviridae WU polyomavirus, Polyomaviridae West Nile virus Flavivirus, Flaviviridae Yaba monkey tumor virus Orthopoxvirus, Poxviridae Yaba-like disease virus Orthopoxvirus, Poxviridae Yellow fever virus Flavivirus, Flaviviridae Zika virus Flavivirus, and Flaviviridae.

Bacterial genera and species detectable by the disclosed biosensor include, but are not limited to, bacteria associated with Xanthomonas, Pseudomonas, Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae, as well as bacterial species such as: P. aeruginosa; E. coli, P. cepacia, S. epidermis, E. faecalis, S. pneumonias, S. aureus, N meningitidis, S. pyogenes, Pasteurella multocida, Treponema pallidum, and P. mirabilis. Gram-negative bacterial genera and species detectable by the disclosed biosensor include, but are not limited to, Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Citrobacter spp., Chlamydia spp., Brucella spp., Pseudomonas spp., Helicobacter spp. and Vibrio spp. Gram-negative bacterial genera and species detectable by the disclosed biosensor include, but are not limited to, Salmonella, E. coli, Yersinia pestis, Klebsiella and Shigella, Proteus, Enterobacter, Serratia, and Citrobacter.

Fungi detectable by the disclosed biosensor include, but are not limited to, fungi associated with Cryptococcus neoformans; Blastomyces dermatitidis; Aiellomyces dermatitidis; Histoplasma capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia; Sporothrix schenckii, Paracoccidioides brasiliensis; Pseudallescheria boydii, Torulopsis glabrata; Trichophyton species, Microsporum species and Dermatophyres species, as well as any other yeast or fungus now known or later identified to be pathogenic.

Parasites detectable by the disclosed biosensor include, but are not limited to, parasites associated with Anaplocephala, Ancylostoma, Necator, Ascaris, Brugia, Bunostomum, Capillaria, Chabertia, Cooperia, Cyathostomum, Cylicocyclus, Cylicodontophorus, Cylicostephanus, Craterostomum, Dictyocaulus, Dipetalonema, Dipylidium, Dracunculus, Echinococcus, Enterobius, Fasciola, Filaroides, Habronema, Haemonchus, Metastrongylus, Moniezia, Nematodirus, Nippostrongylus, Oesophagostomum, Onchocerca, Ostertagia, Oxyuris, Parascaris, Schistosoma, Strongylus, Taenia, Toxocara, Strongyloides, Toxascaris, Trichinella, Trichuris, Trichostrongylus, Triodontophorus, Uncinaria, Wuchereria, Leishmaniasis disease, human African trypanosomiasis disease, Chagas disease, antigens derived from members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.: Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as species including Plasmodium falciparum, P. vivax, P. ovale, P. malaria; Toxoplasma gondii; Leishmania mexicana, L. tropica, L. major, L. aethiopica, L. donovani, Trypanosoma cruzi, T brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T. saginata, Trichomonas vaginitis, T. hominis, T. tenax; Giardia lamblia; Cryptosporidium parvum; Pneumocytis carinii, Babesia bovis, B. divergens, B. microti, Isospora belli, L. hominis; Dientamoeba fragilis; Onchocerca volvulus; Ascaris lumbricoides; Necator americans; Ancylostoma duodenale; Strongyloides stercoralis; Capillaria phihppinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirlus pubis; and Dermatobia hominis, as well as any other parasite now known or later identified to be pathogenic.

Additional pathogens detectable by the disclosed biosensor include, but are not limited to, Coronaviridae (e.g. MERS, SARS-CoV-2), Bunyavirales (e.g. Lassa, Junin, Rift Valley Fever Virus, Andes, Sin Nombre, LaCrosse, California Encephalitis, Crimean Congo Hemorrhagic Fever), Filoviruses (e.g. Ebola, Marburg), Flaviviruses (e.g. Dengue, Zika, West Nile), Paramyxoviridae (e.g. Nipah, Hendra), Picornaviridae (e.g. EV-D68, EV-A71), Togaviridae (e.g. Chikungunya, EEE, VEE, WEE), Bacillus anthracis (including genotypic resistance markers), Yersinia pestis (including genotypic resistance markers), Francisella tularensis (including genotypic resistance markers), Burkholderia spp. (including genotypic resistance markers), Botulinum toxin (including identifying and distinguishing relevant serotypes), ESKAPE pathogens including genotypic resistance markers (e.g., Enterococcus faecium, Staphylococcus aureus, Klebsiella pneumonia, Acinetobacter baumannii, Pseudomonas aeruginosa, Enterobacter spp), Lassa virus, Nipah virus, Rift Valley Fever virus, Enterovirus D68 virus, Candida auris, Coccidioides sp., and novel coronaviruses.

Immuno-based biosensor and environmental detection of airborne pathogens.

In exemplary embodiments, the biosensor will be deployed in an environmental detector for rapid (e.g., real-time or near real-time), continuous measurement of sampled air. The environmental biosensor of the present disclosure for detecting target organisms is surprisingly and unexpectedly based on an ultra-sensitive electrochemical technology used in vivo (e.g., brain, tissue, interstitial fluid, etc.) for Alzheimer's disease research for detecting macromolecular targets.

An initial micro-immunoelectrode (MIE) biosensor was developed to detect amyloid-β (Aβ) peptide in the setting of Alzheimer's disease. The electrochemical sensor uses voltammetry to measure oxidation of tyrosine amino acids within a protein. Oxidation is the release of electrons that the carbon fiber electrode detects as a change in current. The amount of current is proportional to the amount of protein present. The biosensor uses an antibody covalently attached to the surface to provide specificity and concentrate the protein at the electrode for measurement.

The biosensor of the present disclosure is based on a similar design as the MIE. Some embodiments herein describe a CoV-2 nanobody (raised in llama) with 5 nM affinity for the SARS-CoV-2 repeat binding domain (RBD) of the spike protein and very high selectivity over the CoV-1 spike protein. The present disclosure demonstrates that the CoV-2 biosensor has an initial sensitivity of 2 fg/ml. In contrast, conventional antigen tests for CoV-2 are sensitive to the low pg/ml range. Development of the environmental sensor, biosensor, and methods disclosed herein included mimicking real-world environmental conditions, especially in the context of atmospheric aerosols, necessary for testing and optimizing the biosensor's performance for field deployment.

As disclosed herein, an immuno-based electrochemical biosensor provides real-time and continuous measures of CoV-2 aerosols for use in airborne environmental detection and diagnostics. In some embodiments, the sensor targets CoV-2. In other embodiments, design and methodology is adapted to numerous pathogens present in the air or from respiration. The airborne detector described herein monitors gathering spaces for environmental risks to flag for evacuation and/or enhanced disinfection.

In an exemplary embodiment, a CoV-2 biosensor has reasonable sensitivity for recombinant spike protein, and is adaptable depending on viral (pathogen) particles, antibody type, longevity, concentration, and orientation on the electrode surface. In some embodiments, the biosensor is applicable for inactivated CoV-2 viral particle detection. In these embodiments, specificity controls include surface proteins and viral particles of other viruses. Depending upon the embodiment, electrode design is optimized for the size and type of material having the largest impact on specificity and oxidation properties.

Airborne transmission of CoV-2 is caused by the dissemination of droplet nuclei (aerosols) that remain infectious when suspended in air over long distances and time. Guided by observational studies of CoV-2 and other infectious viruses, the biosensor performance metrics of the present disclosure are systematically evaluated with respect to sensitivity, detection limits, and longevity, and aerosolized viral particles are subjected to relevant environmental parameters, including relative humidity, temperature, and atmospheric residence time. The biosensor's performance also includes testing under conditions mimicking real-world indoor and urban atmospheres wherein aerosolized virus droplets are mixed with particulate matter pollutants, such as volatile organics, dust and soot. In some embodiments, an environmental sensor device sampling a given air space detects CoV-2 in real-time or near real-time over a period of at least about 12 hours to about 24 hours.

### Diagnostic Testing.

As noted above, increased and improved ability to test for pathogens, including CoV-2, is needed. While detection and diagnosis of both symptomatic and asymptomatic individuals is needed to inform individual isolations and/or quarantines and to reduce community spread, an equally important, but underdeveloped measure is to monitor a gathering area in real-time (or near real-time) for airborne virus that could result in the shutdown of a space or warrant intense disinfection of the area. In some embodiments described herein, an airborne pathogen sensor is used in conjunction with an aerosol disinfectant mister for immediate cleansing to limit spread of the detected virus and/or pathogen.

### Immuno-Biosensor for Aerosolized and Airborne Detection.

Disclosed herein is an ultra-sensitive immuno-based electrochemical biosensor to detect pathogens, and in exemplary embodiments, the spike protein on the surface of CoV-2. The CoV-2 biosensor disclosed herein is applicable for detecting airborne viruses (and/or pathogens) using an environmental sampling and detection system that can be applied to a large area, such as an airport, hospital, conference center, or school setting. Depending upon the embodiment, a specifically optimized biosensor is implemented to account for conditions of deployment and longevity of sampling and surveillance. Depending upon the embodiment, the collection platform can be modified to detect other pathogens and/or combinations of pathogens.

Innovation of the systems, methods, and devices disclosed herein is based at least in part on the immuno-based biosensor, the nanobody to provide specificity, and/or the sample collection and processing of aerosolized pathogen particles (e.g., CoV-2 viral particles) to real-world environmental conditions prior to testing on the biosensor.

### CoV-2 Biosensor.

Micro-immunoelectrode (MIE) technology as disclosed herein uses square wave voltammetry to measure oxidation of tyrosine amino acids in specific proteins. In embodiments of the CoV-2 biosensor, the biosensor sensitivity has been observed down to 2 fg/ml of CoV-2 spike RBD protein, which in contrast to conventional CoV-2 immunosorbent assays in the low pg/ml range. In some embodiments, the biosensor uses recombinant spike protein. In other embodiments, the biosensor uses CoV-2 viral particles.

As disclosed herein, specificity for a target is based on an antibody covalently attached to the electrode surface. Oxidation of the CoV-2 spike protein bound to the antibody was measured as a direct measure that protein is present. Importantly, tyrosine oxidation is irreversible, meaning the protein bound to the antibody on the surface of the electrode will only be measured once. This is in contrast to many conventional electrochemical sensors that measure impedance at the electrode surface; essentially measuring the binding event instead of the actual protein. Impedance measures can be fraught with specificity issues since non-specific proteins or molecules can deposit on the surface of the electrode and also produce a signal, often referred to as “fouling”.

### Anti-CoV-2 Nanobody.

Anti-CoV-2 nanobodies were obtained from the camelid family (that includes llamas) which produce subclasses of IgGs possessing an unpaired heavy-chain variable domain, known as a nanobody. The nanobodies designed and described herein have been sequenced so they can be grown quickly and cheaply in bacteria for large-scale production, as well as be modified by recombinant molecular biology, e.g. to increase affinity or to orient them on the electrode surface, if needed. Nanobodies are generally hardier than antibodies; withstanding dehydration and larger temperature ranges, which could vary between sampling environments of the airborne detector described herein in conjunction with the biosensor electrode. Airborne detection under realistic environmental conditions.

Aerosol transmission is an important transmission pathway of CoV-2 on the basis of clinical observations in confined spaces. At present, there is a knowledge gap regarding the aerodynamic characteristics and transmission pathways of CoV-2 in aerosols because of challenges associated with their sampling in real-world settings and their quantification at variable particle sizes and concentrations. These real-world factors have direct impact on viral integrity and ability to be measured. Biosensor characterization includes a wide range of virus aerosol size and concentrations, including but not limited to different size distributions of virus aerosols corresponding to the different modes of airborne release via speaking, coughing, and sneezing. Aerosol samples are injected into an environmental chamber to mimic their fate and transport in a real-world indoor environment with pollutants. The biosensor development described herein encompasses a transformational change in the understanding of how environmental conditions alter airborne CoV-2 viral particles.

### CoV-2 Detectors.

The CoV-2 immuno-based biosensor provides ultra-sensitivity for real-time pathogen detection. In some embodiments, the sensor detects CoV-2. Other embodiments include similar sensors developed with antibodies for other pathogens in a multi-electrode array. The airborne detector will enable continuous, instant feedback of a viral threat within the environment. It could flag an area for evacuation or increased disinfection later. It could also be coupled to a disinfectant aerosol spray for immediate resolution in order to keep crowds safe in real-time and limit disruption to on-going activities. Importantly, an airborne detector would alert that someone within a crowd is positive for COVID-19, possibly warranting individual testing within the group to identify and isolate on a much larger scale than currently is possible.

### Environmental Detection of Airborne CoV-2.

SARS-CoV-2 transmits via several modes, including aerosols and droplets, which remain suspended in air long enough to be inhaled. When aerosols or droplets containing respiratory fluid and microorganisms are expelled into unsaturated air, or air with relative humidity (RH) under 100%, they partially or fully evaporate to equilibrate with ambient conditions. This process decreases the particle size and consequently increases its airborne lifetime. Evaporation also increases the concentration of free H+ ions in an aerosol, which in turn, reduce the pH, while solutes such as salts and proteins remain intact. Interactions among salts, changing pH, temperature, and RH in a shrinking droplet are dynamic. Enveloped viruses, such as SARS CoV-2, that partition on the surface of aerosols may be subject to damage from increasing surface tension, shear stress, and conformational rearrangement driven by this dynamic interplay. Unfolding of peptides and subsequent denaturing of proteins can occur at the droplet's air-water interface. Therefore, proper consideration of environmental parameters such as RH and temperature is necessary in determining biosensor design, particularly because they affect the virus transmission dynamics (e.g. size, residence time, distance traveled) and their subsequent detection after they are expelled from infected individuals in the aerosol phase. The recovery and measurement of virus encapsulated in aerosol phase under different environmental conditions is significant for building a device that detects viral particles (e.g., CoV-2 viral particles) in a range of environments and conditions.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. It is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Biosensor

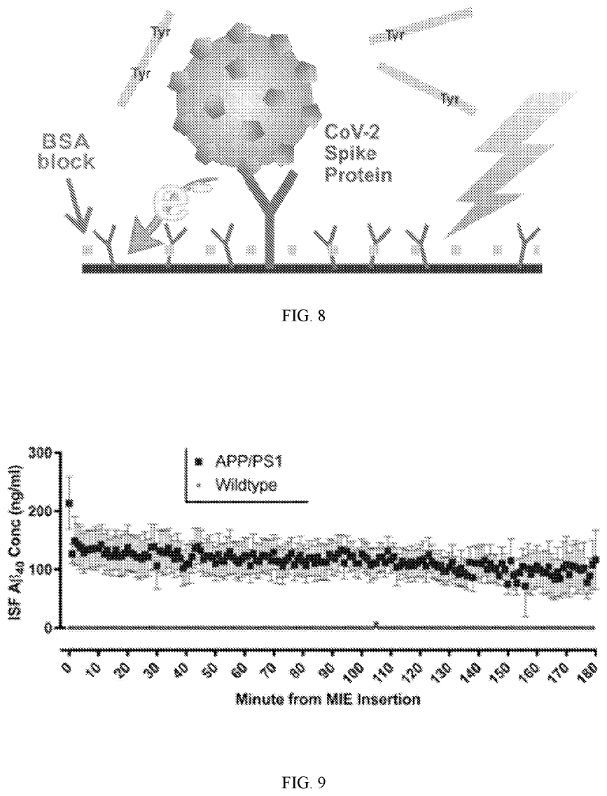

Electrochemical biosensor. Described herein is a micro-immunoelectrode (MIE) technology that uses square wave voltammetry to measure oxidation of tyrosine amino acids (at ~0.65 V) in specific proteins (i.e., pathogen-indicating proteins). Tyrosine oxidation releases electrons that a carbon electrode detects as current (FIG. 8). The amount of current is directly proportional to the amount of analyte present. An antibody covalently attached to the electrode surface concentrates the target at the biosensor for measurement. In the case of SARS-CoV-2, a nanobody (produced in llamas then sequenced and grown cost effectively in bacteria) is covalently attached to the biosensor surface to provide specificity for the biosensor. In some embodiments, one or more nanobodies are attached to the biosensor, alternative or additional to a SARS-CoV-2 nanobody. In embodiments where more than one pathogen is detectable using a single biosensor, a signal (e.g., current) signal from the biosensor may be a multiplexed signal. Importantly, tyrosine oxidation is irreversible, meaning the protein bound to the nanobody on the surface of the electrode will only be measured once. This contrasts with many electrochemical sensors that measure impedance at the electrode surface; essentially measuring the binding event instead of the actual protein. The disclosed biosensor uses screen-printed, inexpensive, carbon-based electrodes (SPiCE).

Amyloid-β (Aβ) micro-immunoelectrode (MIE) of Alzheimer's disease studies. As an example of previous use of similar biosensors, the immuno-based voltametric approach was developed as the micro-immunoelectrode (MIE) biosensors to be used for minute-to-minute measures of human amyloid-β (Aβ) peptide levels in the brain of mouse models of Alzheimer's disease. The Aβ biosensors are surgically implanted into the mouse brain, enabling real-time measurement of the brain interstitial fluid in mice that are awake and freely moving. While the Aβ and SARS-CoV-2 designs are different (5 μm carbon fiber pulled in glass versus a 1 mm screen printed electrode, respectively) based on their intended uses, the principle underlying the biosensors is analogous.

The Aβ biosensor was implanted into the brains of 1) APP/PS1 transgenic mice that express human Aβ or 2) wild-type mice that only express endogenous murine Aβ. Importantly, murine Aβ lacks the tyrosine amino acid in human Aβ that, according to the theory of how the biosensors work, is required to produce the electrochemical signal on the biosensor, serving as a powerful control for specificity in vivo. In APP/PS1 mice, the biosensor measured human Aβ every 60 seconds for 3 hours with minute-to-minute variability that is expected based on on-going neuronal activity (FIG. 9). In contrast, signal in the wild-type mice was negligible for the entire 3-hour measurement period. The Aβ biosensor is 8,000-fold more selective for human Aβ than any other tyrosine in the brain.

A series of biosensors were developed for use in a variety of mouse models of neurological disease, including targeting various species of Aβ peptide (Aβ₄₀, Aβ₄₂, and Aβ oligomers), tau, and α-synuclein. Another MIE was also developed against met-enkephalin, a neuromodulator peptide. Standard Aβ oligomer ELISAs are generally sensitive to the low pg/ml range, whereas the biosensor is sensitive to 200 attogram/ml levels of oligomers, an approximate 10,000-fold increase in sensitivity.

### Example 2. Design of a Breath Aerosol Collection Device

The breath aerosol collection device (or box) has a cap with an inlet straw, and two liquid injection ports. The aerosols from exhaled breath are gathered in a condensing chamber which comprises the upper chamber of the box and consists of a tapered inclined hydrophobic polyimide condensing surface supported by a scaffold. The hydrophobic condensation surface is formed using polyimide high-temperature masking tape. The collection device is stored in a −20 °C freezer prior to running trials to cool the condensing surface. If a freezer is unavailable, a cold fluid like ice water can be added to the box's lower chamber through inlet points on the outer surface of the collection device. When a person exhales into the device, the aerosols impact and condense on the chilled condensing surface, along with any viral particles that may be present. The tapered inclined, hydrophobic surface allows the exhaled breath condensate (EBC) to slide down and settles at the bottom corner of the box, where the micro-immunoelectrode (MIE) biosensor is located. Following the collection of EBC, phosphate buffered saline (PBS) solution is manually injected through the injection ports on the cap. PBS solution is added to wash the any EBC sample still remaining on the inclined surface onto the biosensor at the bottom. Finally, after the EBC is analyzed by the biosensor, hypochlorous acid (HOCl) is injected through the second liquid injection port on the cap to sterilize the breathalyzer for its safe disposal.

### Example 3. Micro-Immunoelectrode (MIE) Biosensor

The electrochemical biosensor uses inexpensive, screen-printed, carbon-based electrodes (SPiCE, Catalog #SP-1401, BASi Research Products, West Lafayette, IN). The core technology for detection of SARS-CoV-2 virions from EBC is based on a micro-immunoelectrode (MIE) technology. SPiCEs are pre-treated in PBS (pH 7.4) and electro-activated using high frequency cyclic voltammetry and chronoamperometry to enhance selectivity for tyrosine oxidation and increase attachment of a SARS-CoV-2 specific nanobody. The nanobody is produced in llamas and is covalently attached to the electrode surface to concentrate the target at the MIE biosensor for measurement. During the prototype phase, the EBC samples were diluted in a cut glass vial containing 1% bovine serum albumin (BSA) in PBS solution rather than analyzed directly in the breathalyzer box. The SPiCE is connected to a commercial potentiostat (PalmSens4, PalmSens BV, Houten, Netherlands). The SPiCE is suspended in the sample vial so that the working electrode is completely submerged to avoid drying out. Square wave voltammetry (SWV) is performed to oxidize tyrosines in the spike protein and detect current change at the electrode surface. In SWV, current at the working electrode is measured while the electrode potential is scanned through 0 to 1 V using a frequency of 15 Hz. When the electroactive species is oxidized, a peak in oxidation current is observed in the voltammogram, which corresponds to the oxidation potential of that particular species. The presence of antibodies covalently attached to the electrode surface provide specificity to SARS-CoV-2 at a potential of 0.65 V. Tyrosine amino acids bear a phenolic group which is easily oxidized at an electrode surface using voltammetry. Voltametric studies of the electrochemical behavior of tyrosine in solution have found tyrosine oxidation to be a 1:1 proton coupled electron transfer process or a 2-electron process. However, the protein scaffold is involved in the electron transfer process, and the oxidation of tyrosine residues within proteins using carbon-based electrodes have been reported to range from 2-4 electrons. Therefore, when the electrode potential is scanned through 0.6V, tyrosine residues in viral particles at the electrode surface will oxidize, releasing up to 4 electrons/molecule that the MIE will detect as current. Although the nanobody recognizes the spike protein, a large portion of the virus is oxidized meaning that tyrosines in many other proteins or the virus surface also release electrons causing signal amplification which likely contributes to the ultra-sensitivity of the sensor.

### Example 4. Materials and Methods

The materials and methods of this example are used in Examples 5-7.

### Cells and Viruses.

Vero cells expressing human ACE2 and TMPRSS2 (Vero-hACE2-hTMPRSS2) were cultured at 37 °C in Dulbecco's Modified Eagle medium (DMEM) supplemented with 10% fetal bovine serum (FBS), 10 mM HEPES (pH 7.3), 100 U/mL of Penicillin-Streptomycin, and 10 μg/mL of puromycin. Vero cells expressing TMPRSS2 (Vero-hTMPRSS2) were cultured at 37 °C in Dulbecco's Modified Eagle medium (DMEM) supplemented with 10% fetal bovine serum (FBS), 10 mM HEPES (pH 7.3), 100 U/mL of Penicillin-Streptomycin, and 5 μg/mL of blasticidin.

The original strain of SARS-CoV-2 (strain 2019-nCoV/USA-WA1/2020), and the Delta (B.1.617.2) and Omicron (B.1.1.529) variant of SARS-CoV-2 were propagated on Vero-hTMPRSS2 cells. The infectious virus titer was determined by plaque assay on Vero-hACE2-hTMPRSS2 cells.

In order to inactivate SARS-CoV-2, culture supernatant containing infectious virus, was treated for 18 hours with 1:1000 dilution of beta-Propiolactone (BPL). Following the inactivation of BPL at 37 °C for one hour, the inactivation of SARS-CoV-2 was confirmed by plaque assay on Vero-hACE2-hTMPRSS2 cells as reported previously. An inactivated sample and a positive control were included in the assay.

### Laboratory Aerosolization Experiments.

The experimental setup for collection of EBC sample includes the CHεST® (CH Technologies (USA), Inc) and the breath aerosol analyzer. CHεST® is a device which can simulate the conditions and particle production corresponding to both respiratory exhalation and cough/sneeze. It includes a Blastein Atomizing Module (BLAM, CH Technologies (USA), Inc) that simulates the aerosol size distributions generated during various respiratory activities, such as breathing and sneezing. The BLAM is a series of atomizers that generate aerosols using the jet nebulization principle. The cycle period, i.e., the period of aerosol generation and cycle duration, i.e., interval between consecutive cycles are defined by the user. To simulate a person breathing into the device, the BLAM was placed outside the jar. The cycle period and interval are set to 5 seconds each in order to simulate continuous exhalation conditions. In order to estimate the flow rate of compressed air that passes through the atomizer, the following calculations are done: The aerosolization experiments mimic 10-15 deep breaths by a person. The average rate of breathing when a person is resting is 7-8 LPM and the forced expiratory volume (FEV) is typically 0.5 L. However, in deep breathing, the FEV can vary between 60-80% of the total lung capacity (6 L) depending on the gender and age of person. A FEV of 4.8 L during deep breathing was estimated, and hence the total expiratory volume during 10-15 deep breaths is in the range of 48-70 L. Hence, compressed air at a flow rate of 5.5 LPM (20 psi pressure setpoint) is sent to the CHεST® and sample collection is done for a period of 10 min. Additionally, the CHεST® is paired with a syringe pump for efficient fluid delivery. Inactivated virus solution (100 μL inactivated SARS-CoV-2 virus in 25 mL PBS) is passed through the syringe pump to the BLAM at a flow rate of 0.9 mL/min. Thus, the inlet to the BLAM atomizer includes compressed air and the inactive virus solution.

When the setup is switched on, the atomizer generates aerosols which travel to the breathalyzer device. The BLAM simulates the aerosols production and conditions corresponding to forced exhalation. There is a conical attachment with an extended cylindrical body that connects the outlet of the atomizer to the input straw of the breathalyzer device. The 3D-printed conical attachment helps constrict the path of aerosols generated and results in greater impaction on the breathalyzer surface. The breathalyzer is kept in a −20 °C freezer for at least 10 minutes min before each trial. There is a temperature difference between the stream of aerosols generated through the atomizer (higher temperature) and the chilled surface of the collection device (lower temperature) which causes the impacted aerosol particles to condense. Due to the inclined and hydrophobic nature of the cold surface, the condensate slides to the bottom of the collection device. At the end of the 10 min period, the EBC sample condensed on the surface of the breathalyzer is collected from the bottom, washed with 1 mL PBS and sent for analysis.

### Electrode Preparation.

The biosensor uses a screen-printed carbon electrode chip. In order to enhance tyrosine oxidation and efficient binding of the nanobody, the working electrodes are pretreated in PBS using a triangular waveform from 0 to 3V at 70 Hz for 20 seconds, followed by holding at −0.8 V and 1.5 V for 5 sec and 10 sec respectively. The activation of carboxylic groups on the electrode surface is achieved by using 0.4 M EDC ((N-(3-Dimethylaminopropyl)-N′-ethylcarbodiimide hydrochloride) and 0.1 M NHS (N-Hydroxysuccinimide) solution (Thermo Scientific, IL, USA) to form a semi-stable reactive amine NHS ester. The activated electrodes are placed in a solution of the nanobody and incubated for 10 min at room temperature, followed by 4 °C overnight. After nanobody is attached to the electrode surface, the biosensors are incubated with 0.05% ethanolamine to deactivate the reactive amine sites and with 0.1% albumin to block non-specific protein binding sites.

### Characterization of Electrochemical Measurements.

Prior to analysis, the EBC samples are diluted in a cut glass vial containing 1% bovine serum albumin (BSA) in PBS solution. The biosensor is connected to a commercial potentiostat (PalmSens4) and SWV is performed in the blank and sample solutions. In SWV, the electrode potential is scanned from 0 to 1 V at a frequency of 15 Hz. The voltammograms are acquired using a handheld potentiostat (PalmSens) with the supplied PSTrace 5.9 software in a three-electrode setup.

### Virus Aerosol Recovery (%).

In lab experiments, a stock solution of the inactivated SARS-CoV-2 particles and PBS is initially prepared. The virus concentration was measured (using RT-qPCR) of the stock solution to determine the virus aerosol recovery (%) of the breath aerosol collector.

The virus aerosol recovery is calculated as a ratio of the viral copies detected in aerosolized samples to the total number of viral copies present in the stock solution, and is expressed as a percentage of the latter.

$$EBC virus aerosol recovery ( \% ) = \frac{Sample RNA Copies}{Total RNA copies ( Theoretical )} \times 100$$

The total theoretical RNA copies depend on stock RNA load and volume of aerosolized sample entering the collection device, and the sample RNA copies depends on the sample RNA load and volume of EBC collected. The EBC sample is diluted with 1 mL phosphate buffer saline (PBS) solution, thus the dilution factor is also accounted for in the calculations.

Total theoretical RNA copies=(Stock viral load)*(volume of aerosolized sample)

$$Dilution factor = \frac{( 1000 μL PBS + Volume of EBC collected ( μL ) )}{Volume of EBC collected ( μL )}$$

Sample RNA copies=(Sample RNA load)×(EBC volume)×(dilution factor)

Thus, the virus aerosol recovery (%) is estimated for the different SARS-CoV-2 variants.

### Predicted Number of Breaths.

The predicted number of breaths is calculated from the limit of detection (LoD) of the MIE biosensor for different SARS-CoV-2 variants. The LoD refers to the minimum virus RNA concentration (copies/ml) for which the biosensor produces a corresponding oxidation current (lox). The typical viral load in 20 exhaled breaths ranges from 70-30,000 copies/mL, with a mean of 2470 copies/mL. This range was used to evaluate the viral copies per breath, and the minimum number of breaths for detection can be subsequently calculated.

RNA copies/breath varies from 3.5 to 1500, with a mean of 123.5.

The MIE biosensor LoD for different variants is shown in the below table.

| | | |
|---|---|---|
| Beta | 8.03 | copies/ml |
| WA1 | 31.8 | copies/ml |
| Delta | 5.94 | copies/ml |
| Omicron | 21 | copies/ml |

Average EBC sample in lab experiments=1.04 mL (including dilution with PBS)

Total RNA copies in sample=(LoD)×(Volume of EBC sample)

$$Minimum exhaled breaths for detection = \frac{Total RNA copies in sample}{RNA copies / breath}$$

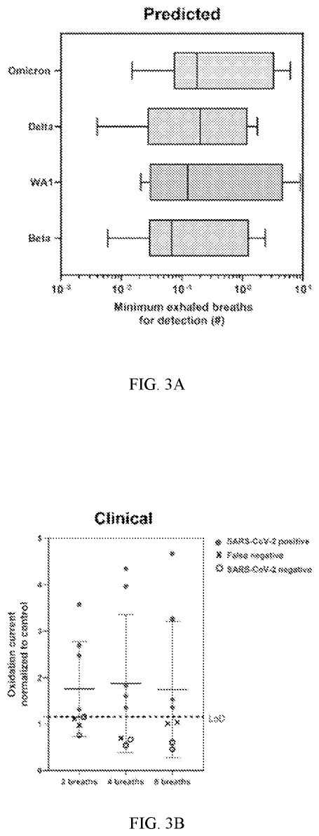

The minimum number of exhaled breaths for biosensor detection varies from 0.04-9 for the different SARS-CoV-2 variants. The box and whisker plots of FIG. 3A are obtained by assuming two additional data points in the range with viral load ±33.33% of the mean (2470 copies/ml).

Detection of SARS-CoV-2 from Clinical Samples.

In order to evaluate the performance of the diagnostic setup in human subjects, 8 participants were tested (n=6 COVID positive and n=2 COVID negative) using the setup in a clinical study. For the clinical study, the design of the breath aerosol collection device is modified to include a slot at the bottom of the box that can fit an Eppendorf tube. Thus, the EBC sample can directly slide down the hydrophobic surface and is collected in the tube. The breathalyzer is kept in a −20 °C freezer for at least 1 hr prior to EBC sample collection. Each participant blows into the breathalyzer 2, 4, and 8 times and the corresponding EBC sample is collected. Thus, 3 samples are obtained from each participant. PBS is added through the inlet port on the cap of the breathalyzer to wash any remaining EBC sample into the Eppendorf tube. After sample collection is done, the breathalyzer unit is disinfected with HOCl and safely disposed. The EBC samples are then analyzed in the laboratory using the MIE biosensor for the presence of SARS-COV-2.

### Example 5. Biosensors

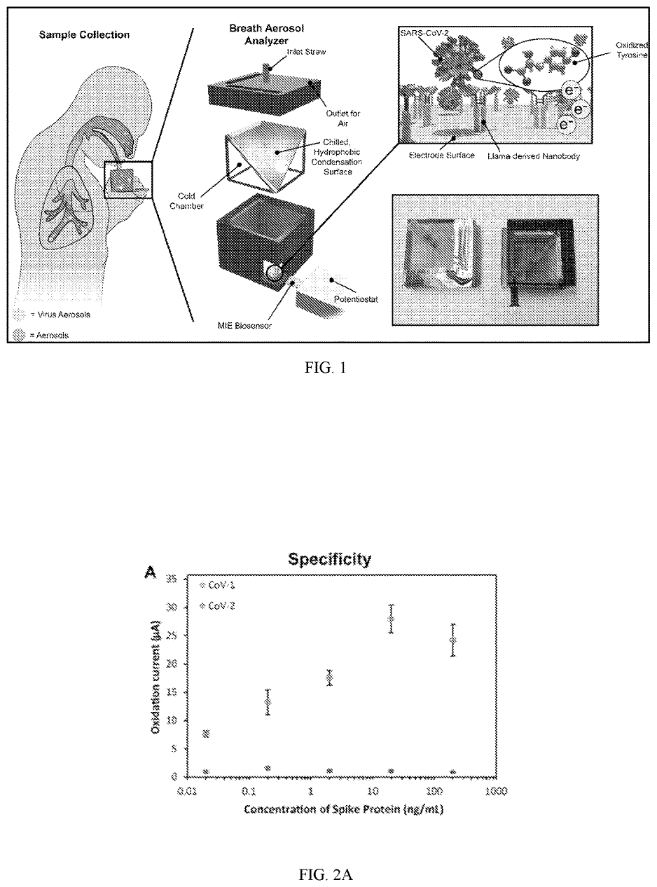

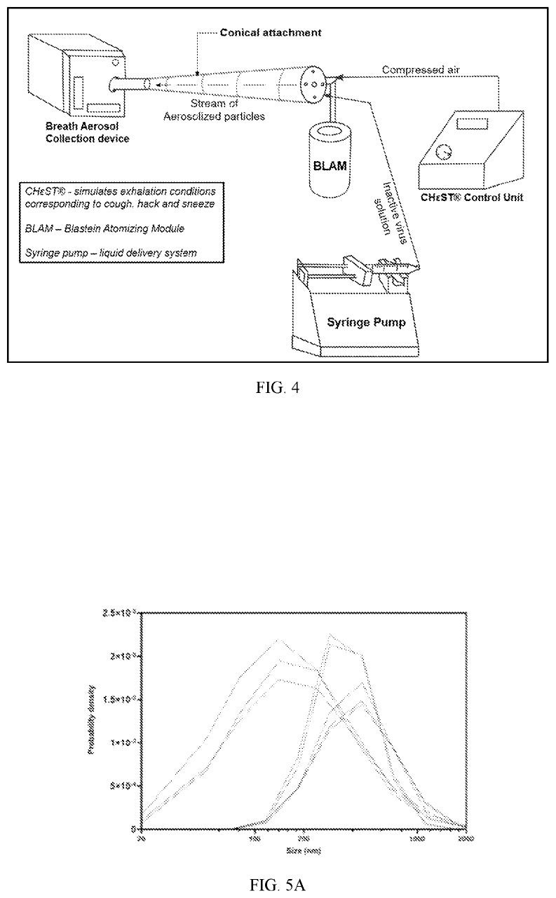

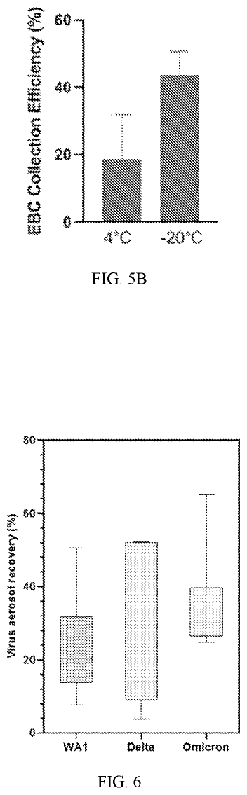

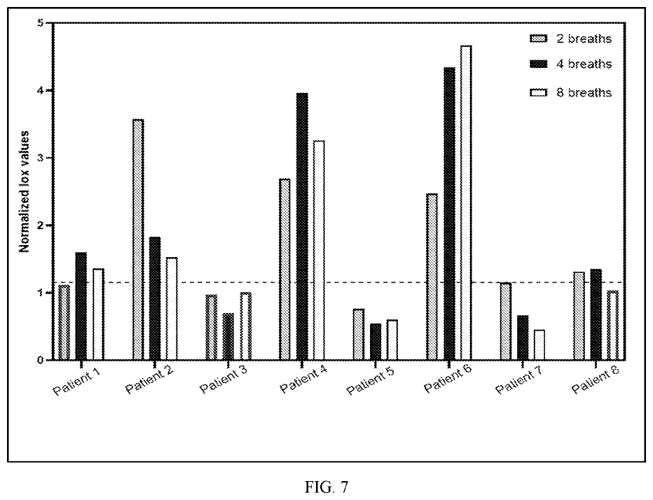

The biosensors described herein combine recent advances in EBC sampling and ultrasensitive electrochemical detection of SARS-CoV-2 variants using llama-derived nanobodies to develop a hand-held, point-of-care breath aerosol analyzer with micro-immunoelectrode (MIE) biosensor for clinical diagnosis. The breath aerosol collector has a detachable inlet straw through which a patient exhales into the device (FIGS. 1 and 4). Virus-laden respiratory aerosols from the warm, exhaled breath impact and condense on the chilled hydrophobic surface. The surface is washed with 1 mL of 1% bovine serum albumin (BSA) in phosphate buffer saline (PBS) along the tapered incline to deliver the condensed aerosols to the bottom corner of the box, where the MIE biosensor is located. The biosensor uses screen-printed carbon-based electrodes with a nanobody originally derived in llamas covalently bound to the electrode surface to provide specificity to SARS-CoV-2 spike protein (FIGS. 5A-5B). The biosensor detects the oxidation of tyrosine amino acids present in the spike protein of SARS-COV-2 (FIG. 1). The surface of the biosensor is pre-blocked in 1% BSA to prevent non-specific binding. Importantly, tyrosine amino acids cannot be reduced to oxidize again, so any tyrosine present in the nanobody or BSA are oxidized in the electrode preparation and so cannot provide signal during the actual test. The MIE biosensor is connected to a commercial potentiostat and square wave voltammetry is performed to oxidize tyrosine and measure the peak oxidation current corresponding to the presence of virus aerosols in a given sample. Tests will be single-use and provide results in under one minute which is an improvement compared to conventional viral diagnostics.

### Example 6. Performance Parameters

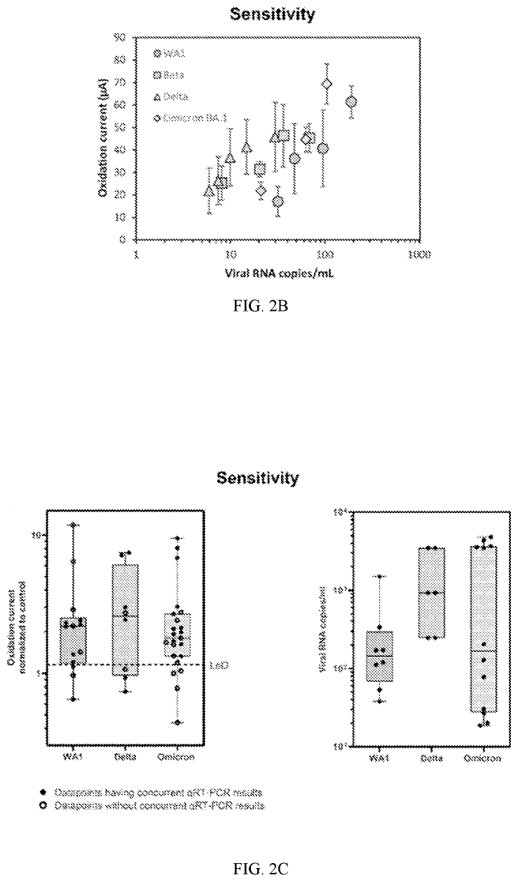

The specificity of the MIE biosensor was evaluated by comparing the peak tyrosine oxidation currents (Iₒₓ) for varying concentrations of SARS-CoV-2 and SARS-CoV-1 spike protein. SARS-CoV-2 produced robust signal down to 20 pg of spike protein per mL of sample fluid and saturates around 20 ng/mL, whereas SARS-CoV-1 produced negligible signal (FIG. 2A). The biosensor is highly specific towards SARS-CoV-2, despite both spike proteins having more than 70% of their genetic makeup in common.

The limit of detection (LoD) of the MIE biosensor was evaluated by sequential dilution of a purified inactivated SARS-CoV-2 stock solution and measuring the corresponding Ix values for different virus concentrations (confirmed using RT-qPCR). The lowest virus RNA concentrations detected by the MIE biosensor were 32, 8, 6, and 21 RNA copies/mL for the USA/WAa1/2020 (WA1), Beta (B.1.351), Delta (B.1.617.2) and Omicron (BA. 1) strains of SARS-CoV-2, respectively (FIG. 2B). The biosensor LoD is equal to or better than comparable sensors. Also, the LoD for all the variants is much lower than typical viral RNA load in exhaled breath of individuals infected with SARS-CoV-2, which highlights the potential of the MIE biosensor for ultrasensitive detection of virus aerosols in exhaled breath. An individual oxidized tyrosine releases two to four electrons that the MIE biosensor detects as current. The sensor response plateaus at higher concentrations for the Beta and Delta variants, likely a result of the Hook effect due to excessive analyte concentration or limited nanobodies on the electrode surface.

To evaluate device performance, inactivated SARS-CoV-2 virions of three different variants: WA1, Delta (B.1.617.2), and Omicron (BA.1) were aerosolized in laboratory experiments. Aerosols were generated that mimic the size distribution of exhaled breath originating from the lower airways of lungs, and the volume of air nebulized corresponds to the expiratory volume from 10-15 “deep” breaths by a person (FIGS. 4 and 5A-5B). Aerosolization runs using pure PBS solution constituted the control for the method. FIG. 2C shows the average Iₒₓ values measured for aerosolized virus sampled in the breath aerosol collector normalized to the Iₒₓ values of the control, indicating a 77.8% sensitivity (n=45) for this device. The sensitivity of the method is comparable to other electrochemical detection techniques for SARS-CoV-2; however, this method focuses on direct detection of virus-laden aerosols and will provide results in under one minute.

The results from RT-qPCR determined that viral RNA for samples collected using the breath aerosol analyzer ranged from 10¹³ to 10^{3.7} gene copies/sample (FIG. 2C). These values are in line with viral loads reported using EBC-based methods, which are approximately 3-4 orders lower in magnitude compared to that from nasal swabs COVID-19 infection results in 200-600 viral particles per breath, which reinforces the feasibility of the method to detect virus aerosols in exhaled breath.

### Example 7. Clinical Evaluation

To validate the performance of the system in human patients, the device was employed in a clinical trial. EBC samples were analyzed from 8 participants (Six COVID positive and two COVID negative, as determined by RT-qPCR of nasopharyngeal swabs) using the CoV-2 EBC. Assuming that the viral load from 20 exhaled breaths ranges from 70 to 30,000 copies/mL (mean=2470 copies/mL) (20), the minimum exhaled breaths for MIE biosensor detection was predicted to vary from 0.05 to 9 for different SARS-CoV-2 variants. This is depicted in FIG. 3A. Thus, the standard sample collection protocol for the clinical trial was defined so that each participant blew into different EBCs two, four, then eight times with approximately three minutes between consecutive sample collection (n=24 samples). The initial results (FIG. 3B) denote a 77.9% sensitivity (n=8 subjects) for the method, and a specificity of 100% as the analyte signal for negative patients is below the LoD. The Ix values also demonstrate that 2 exhaled breaths are sufficient for detection using the MIE biosensor. While the biosensor was tested with known inactivated viral particles in vitro through the BA.1 variant, the clinical study was in Summer to Fall 2022 when the BQ.1 variant was predominant, though the human subjects were not sequenced to determine which variant was present.

### SUMMARY

In summary, demonstrated herein is a portable, point-of-care testing platform integrating a novel breath aerosol collector and a nanobody-based MIE biosensor, costing less than 10 USD for each test which will provide results in under one minute. The sampling technique is non-invasive, and the detection method is rapid, facile and does not warrant the need for highly trained personnel. Additionally, satisfactory results were obtained from just 20 seconds of sampling (2 exhaled breaths) compared to 5-30 minutes of sampling in typical EBC-based studies. Finally, the MIE biosensor is highly sensitive for SARS-CoV-2 detection and has a lower LoD compared to similar devices.

The absolute value of tyrosine oxidation peak current (IU) measured using the biosensor depends on the amount of surface-attached nanobodies and the concentration of the analyte, along with extrinsic factors such as ambient relative humidity and temperature. The variation in individual electrode responses precluded performing a direct comparison of gene copies obtained from RT-qPCR to the Ix values. The consequences of Hook effect at higher viral loads needs further investigation.

The platform is readily adaptable to not only detect different CoV-2 variants, but also other respiratory pathogens of interest.

### CONCLUSIONS

Airborne transmission via virus-laden aerosols is a dominant route for the transmission of respiratory diseases, including SARS-CoV-2. Direct, non-invasive screening of respiratory virus aerosols in patients has been a longstanding technical challenge. Provided herein is a point-of-care testing platform that directly detects CoV-2 aerosols in as little as two exhaled breaths of patients. It integrates a hand-held breath aerosol collector and a llama-derived, CoV-2 spike-protein specific nanobody bound to an ultrasensitive micro-immunoelectrode biosensor, which detects the oxidation of tyrosine amino acids present in CoV-2 spike protein. Results were within 20% of those obtained using standard testing methods. This platform holds the potential to be adapted for multiplexed detection of different respiratory viruses. It provides a rapid and non-invasive alternative to conventional viral diagnostics.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A biosensor comprising:
an activated electrode comprising a nanobody deposited thereon.

2. The biosensor of claim 1, wherein the activated electrode comprises amine-reactive groups on the electrode surface.

3. The biosensor of claim 1, wherein the activated electrode comprises amine-reactive NHS ester groups on the electrode surface.

4. The biosensor of claim 1, wherein the nanobody is bound to the activated electrode by an amide bond.

5. The biosensor of claim 1, wherein the nanobody is bound to a target protein.

6. The biosensor of claim 1, wherein the electrode is a carbon-based electrode.

7. The biosensor of claim 1, wherein the nanobody is a nanobody from the camelid family.

8. The biosensor of claim 1, wherein the nanobody provides specificity and/or concentrates a protein at the electrode.

9. The biosensor of claim 1, wherein the biosensor is configured to perform voltammetry.

10. The biosensor of claim 1, wherein the biosensor is configured to measure oxidation of tyrosine amino acids in a protein.

11. The biosensor of claim 1, wherein the biosensor is configured to analyze an environmental air sample and detect airborne pathogens.

12. The biosensor of claim 1, wherein the biosensor is configured to detect multiple pathogens simultaneously in a single test.

13. The biosensor of claim 1, wherein the biosensor is configured to detect an airborne pathogen selected from the group consisting of viruses, bacteria, parasites, fungi, mold, variants thereof, strains thereof, and combinations thereof.

14. The biosensor of claim 1, wherein the biosensor is configured to detect a repeat binding domain (RBD) of a SARS-CoV-2 spike protein.

15. The biosensor of claim 1, wherein the biosensor is configured to detect at least one variant of SARS-CoV-2.

16. A method for analyzing aerosolized samples from breath or an environmental air sample to detect aerosolized pathogens, the method comprising:
receiving an environmental air sample at a biosensor; and
detecting an airborne pathogen,
wherein the biosensor comprises an activated electrode comprising a nanobody deposited thereon.

17. An airborne detection device for analyzing aerosolized samples from breath or an environmental air sample to detect aerosolized pathogens, the device comprising:
a biosensor comprising an activated electrode comprising a nanobody deposited thereon; and
a sample collection device.

18. The airborne detection device of claim 17, wherein the sample collection device is a breath aerosol collection device.

19. The airborne detection device of claim 17, wherein the airborne detection device is configured to detect multiple pathogens simultaneously in a single test.

20. The airborne detection device of claim 17, wherein the airborne detection device is configured to detect an airborne pathogen selected from the group consisting of viruses, bacteria, parasites, fungi, mold, variants thereof, strains thereof, and combinations thereof.
